(19) United States
(12) Patent Application Publication
(10) Pub. No.: US 2021/0161131 A1
(43) Pub. Date: Jun. 3, 2021

(54) cis-p-Menthane-1,8-Di-secondary Amine Compounds as well as Their Preparation Methods and Application in Weed Control

(71) Applicants: RESEARCH INSTITUTE OF FORESTRY NEW TECHNOLOGY, CHINESE ACADEMY OF FORESTRY, Beijing (CN); INSTITUTE OF CHEMICAL INDUSTRY OF FOREST PRODUCTS, CHINESE ACADEMY OF FORESTRY, Nanjing, Jiangsu (CN)

(72) Inventors: Shichao XU, Nanjing (CN); Zhendong ZHAO, Nanjing (CN); Xiaojing ZENG, Nanjing (CN); Jing WANG, Nanjing (CN); Huanhuan DONG, Nanjing (CN); Hongmei ZHANG, Nanjing (CN); Yuxiang CHEN, Nanjing (CN); Liangwu BI, Nanjing (CN)

(73) Assignees: Research Institute of Forestry New Technology, Chinese Academy of Forestry, Beijing (CN); Institute of Chemical Industry of Forest Products, Chinese Academy of Forestry, Nanjing, Jiangsu (CN)

(21) Appl. No.: 17/053,348

(22) PCT Filed: Apr. 22, 2020
(86) PCT No.: PCT/CN2020/086219

§ 371 Date: Nov. 5, 2020

(30) Foreign Application Priority Data
Jun. 6, 2019 (CN) 201910490796.6

(51) Int. Cl. A01N 33/04; A01N 43/08; A01N 43/10; A01N 43/36; A01N 43/40

(52) U.S. Cl. CPC A01N 33/04; A01N 43/08; C07D 207/335; A01N 43/36; A01N 43/40; A01N 43/10

(57) ABSTRACT

The present invention discloses the preparation method of cis-p-menthane-1,8-di-secondary amine compounds and their application in weed control. The preparation method is as follows: cis-p-menthane-1,8-di-Schiff base compounds are used as the raw material, and the reducing agent is added in batches into the raw material in a polar organic solvent at −10˜50° C. to react for 1˜24 h; after the reaction is complete, the reaction solution is quenched with distilled water and extracted with dichloromethane, and the extract phase is dried with anhydrous sodium sulfate, filtered with a filter and then distilled under reduced pressure to obtain crude cis-p-menthane-1,8-di-secondary amine compounds, which are purified into the pure product through recrystallization or silica gel column chromatography. The Petri dish seed germination method is used to determine the root and stem growth inhibition levels of barnyard grass incubated under different concentrations of cis-p-menthane-1,8-di-secondary amine compounds to evaluate the herbicidal activity of the compounds.

### TECHNICAL FIELD

The present invention relates to cis-p-menthane-1,8-di-secondary amine compounds as well as their preparation methods and application in weed control, specifically relating to a type of cis-p-menthane-1,8-di-secondary amine compounds which are prepared by using cis-p-menthane-1,8-di-Schiff base compounds as the raw material and making the raw material undergo reduction reaction with a reducing agent in a polar organic solvent. Moreover, the present invention also uses cis-p-menthane-1,8-di-secondary amine compounds as the active ingredients of herbicides used to remove barnyard grass and other weeds in paddy fields.

### BACKGROUND TECHNOLOGY

Since the middle of the 20th century, the application of herbicides has been of great significance to the increase of global food production. However, traditional herbicides are mainly organic synthetic compounds and are highly toxic, chemically stable and difficult to biodegrade. Therefore, actively developing green, environment-friendly high-performance herbicides to replace traditional chemically synthesized herbicides has become a research hotspot in the field of pesticide development around the world in recent years.

Plant-derived herbicides are developed with natural plant resources. They are rich in natural sources and are highly effective, low-toxic and biodegradable. They are one of the most widely studied new environment-friendly pesticides in recent years. Monoterpenoids containing a p-menthane (1) or p-menthene (2) skeleton are the most widely distributed terpenoids in nature. Due to their good environmental compatibility and biological activity, the research and application of plant-derived herbicides containing a p-menthane (1) or p-menthene (2) skeleton have attracted more and more attention from scientific researchers all over the world. Such compounds show bright prospects for development and utilization. Among them, cinmethylin (3), a type of oxygen-containing heterocyclic p-menthane derivatives, is one of the plant-derived herbicides having the best known commercial application effect. Cinmethylin can effectively inhibit the growth of plant meristems and has a wide application period, small dosage and other advantages (Pesticides, 1996, 35 (3): 34-34; Chemistry and Industry of Forest Products, 2019, 39(2): 1-8).

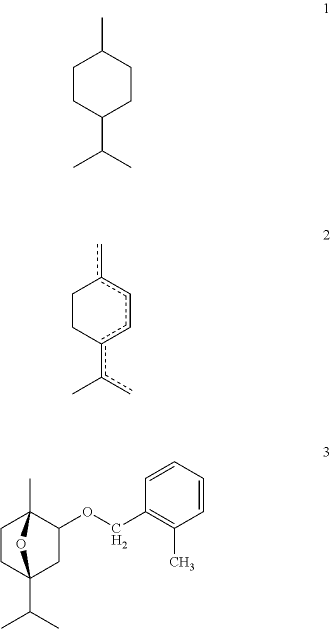

In the completed preliminary research work, a series of cis-p-menthane-1,8-di-Schiff base plant-derived herbicidal active substances were synthesized from turpentine or pinene. Most of the compounds have better inhibitory effects on the root and stem growth of annual ryegrass than commercially available herbicide diuron or glyphosate (ZL 201610942979.3; Chinese Chemical Letters, 2017, 28(7), 1509-1513; Industrial Crops & Products, 2018, 115:111-116). However, Schiff base compounds contain unsaturated imine groups (—C═N—) and are chemically unstable. They are not only easily oxidized by oxygen in the air, but are also prone to hydrolysis and other reactions under acidic conditions. This limits their further development and application. Through the hydrogenation reduction reaction, the imine groups can be reduced to chemically stable secondary amine groups, and the obtained secondary amine groups are also the active centers of a variety of medicinal and pesticidal compounds and can effectively improve the herbicidal activity of related compounds.

At present, the reductive hydrogenation of the —C═N-bond of Schiff base compounds is mainly achieved with chemical reducing agents (Journal of Environmental Science and Health, Part B, 2009, 44, 344-349), but there are relatively few related studies on this issue. Research on the reductive hydrogenation of cis-p-menthane-1,8-di-Schiff base compounds using chemical reducing agents has not been reported yet. The present invention discloses a new type of plant-derived herbicides, that is, cis-p-menthane-1,8-di-secondary amine compounds, as well as their preparation methods and application in the field of weed control.

### SUMMARY OF THE INVENTION

In order to overcome the shortcomings of synthetic pesticides such as high toxicity and difficult biodegradation, the present invention discloses a new type of plant-derived herbicides—cis-p-menthane-1,8-di-secondary amine compounds, as well as their preparation methods and application in weed control. The present invention uses cis-p-menthane-1,8-di-Schiff base compounds as the raw material and reduces imine groups in the raw material to chemically stable secondary amine groups through the hydrogenation reduction reaction to produce cis-p-menthane-1,8-di-secondary amine compounds. Moreover, it measures the herbicidal effect of the resulted compounds on barnyard grass in paddy fields through the Petri dish seed germination method.

The technical solution of the present invention is: cis-p-menthane-1,8-di-secondary amine compounds, whose general structure is as follows:

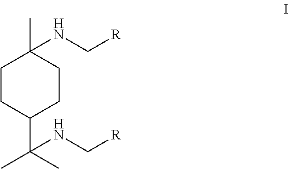

In general structure I, R is any one of

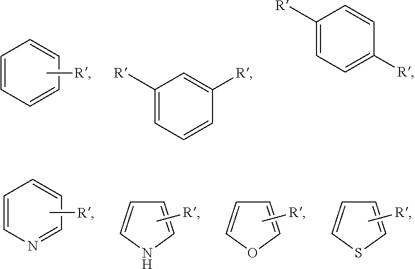

and R′ is any one of hydrogen, alkyl, substituted alkyl or halogen groups.

The preparation method of the above-mentioned cis-p-menthane-1,8-di-secondary amine compounds is to use cis-p-menthane-1,8-di-Schiff base compounds as the raw material, dissolve the raw material in a polar organic solvent and then add a reducing agent into the resulted solution in batches to have reduction reaction. After the reaction finishes, the reaction solution is subject to post-treatment to obtain a pure product.

The above-mentioned polar organic solvent is any one of methanol, ethanol, tetrahydrofuran, dimethylformamide and dimethyl sulfoxide, or a mixture of several of them.

The above-mentioned reducing agent is any one of sodium borohydride, potassium borohydride and lithium aluminum hydride.

The molar ratio of cis-p-menthane-1,8-di-Schiff base compounds to the reducing agent is between 1:1-5.

The reaction temperature is between −10˜50° C.

The reaction time is 1˜24 h.

The above-mentioned post-treatment specifically refers to quenching the reaction solution with distilled water, extracting it with dichloromethane, drying the extract phase with anhydrous sodium sulfate, filtering the extract phase, obtaining crude cis-p-menthane-1,8-di-secondary amine compounds through vacuum distillation, and turning the crude product into the pure product through recrystallization or silica gel column chromatography.

The above-mentioned cis-p-menthane-1,8-di-secondary amine compounds are used as active ingredients of herbicides.

The above-mentioned cis-p-menthane-1,8-di-secondary amine compounds are used as active ingredients of herbicides for controlling barnyard grass in paddy fields.

### BENEFICIAL EFFECTS

The cis-p-menthane-1,8-di-secondary amine compounds disclosed by the present invention are the derivatives of natural products and belong to monoterpenoids plant-derived herbicidal active substances. They are characterized by low toxicity, high safety and environmental friendliness.

The synthesis process disclosed by the present invention is simple and has mild conditions, fast reaction rate, high product yield and wide substrate applicability, and is suitable for mass production.

The cis-p-menthane-1,8-di-secondary amine compounds disclosed by the present invention have excellent inhibitory effects on the root growth of barnyard grass in paddy fields and have higher herbicidal activity than the corresponding cis-p-menthane-1,8-di-Schiff base compounds.

### BRIEF DESCRIPTION OF THE DRAWINGS

FIG. 1 is the ¹H nuclear magnetic resonance (¹H NMR) chart of cis-p-menthane-1,8-bis-(4-chlorobenzyl)amine;

FIG. 2 is the ¹H NMR chart of cis-p-menthane-1,8-bis-(4-fluorobenzyl)amine;

FIG. 3 is the ¹H NMR chart of cis-p-menthane-1,8-bis-(4-bromobenzyl)amine;

FIG. 4 is the ¹H NMR chart of cis-p-menthane-1,8-bis-(4-methylbenzyl)amine;

FIG. 5 is the ¹H NMR chart of cis-p-menthane-1,8-bis-(4-methoxybenzyl)amine;

FIG. 6 is the ¹H NMR chart of cis-p-menthane-1,8-bis-(4-trifluoromethylbenzyl)amine;

FIG. 7 is the ¹H NMR chart of cis-p-menthane-1,8-bis-(4-dimethylaminobenzyl)amine;

FIG. 8 is the ¹H NMR chart of cis-p-menthane-1,8-bis-(4-methylthiobenzyl)amine;

FIG. 9 is the ¹H NMR chart of cis-p-menthane-1,8-bis-(2-trifluoromethylbenzyl)amine;

FIG. 10 is the ¹H NMR chart of cis-p-menthane-1,8-bis-(5-bromofuran-2-methyl)amine;

FIG. 11 is the ¹H NMR chart of cis-p-menthane-1,8-bis-(5-methylfuran-2-methyl)amine;

FIG. 12 is the ¹H NMR chart of cis-p-menthane-1,8-bis(furan-2-methyl)amine;

FIG. 13 is the ¹H NMR chart of cis-p-menthane-1,8-bis-(pyridine-2-methyl)amine;

FIG. 14 is the ¹H NMR chart of cis-p-menthane-1,8-bis-(pyridine-3-methyl)amine;

FIG. 15 is the ¹H NMR chart of cis-p-menthane-1,8-bis-(pyridine-4-methyl)amine;

FIG. 16 is the ¹H NMR chart of cis-p-menthane-1,8-bis-(thiophen-2-methyl)amine;

FIG. 17 is the ¹H NMR chart of cis-p-menthane-1,8-bis-(pyrrole-2-methyl)amine.

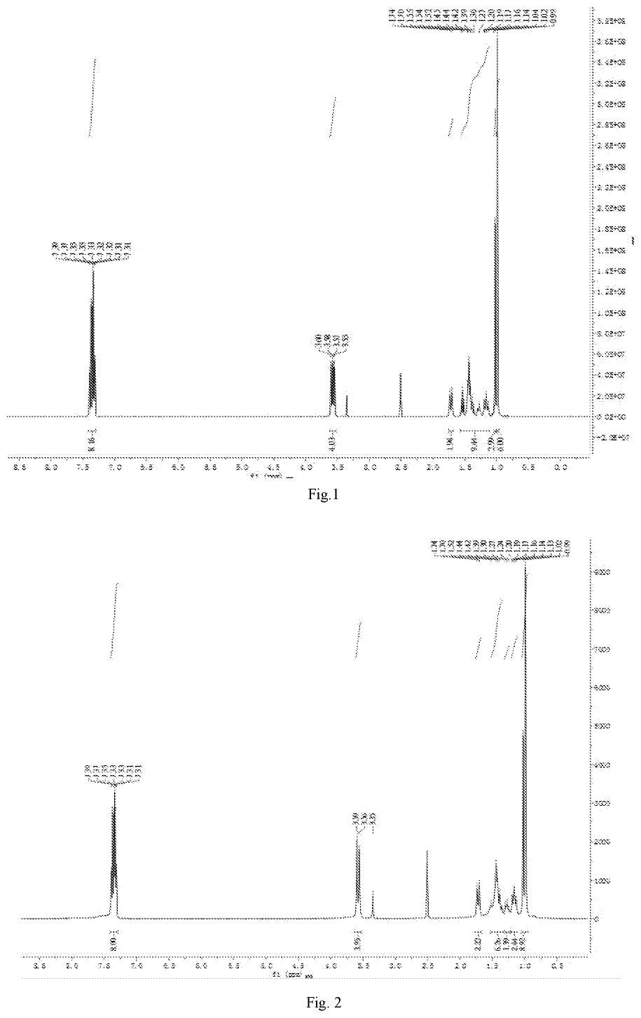

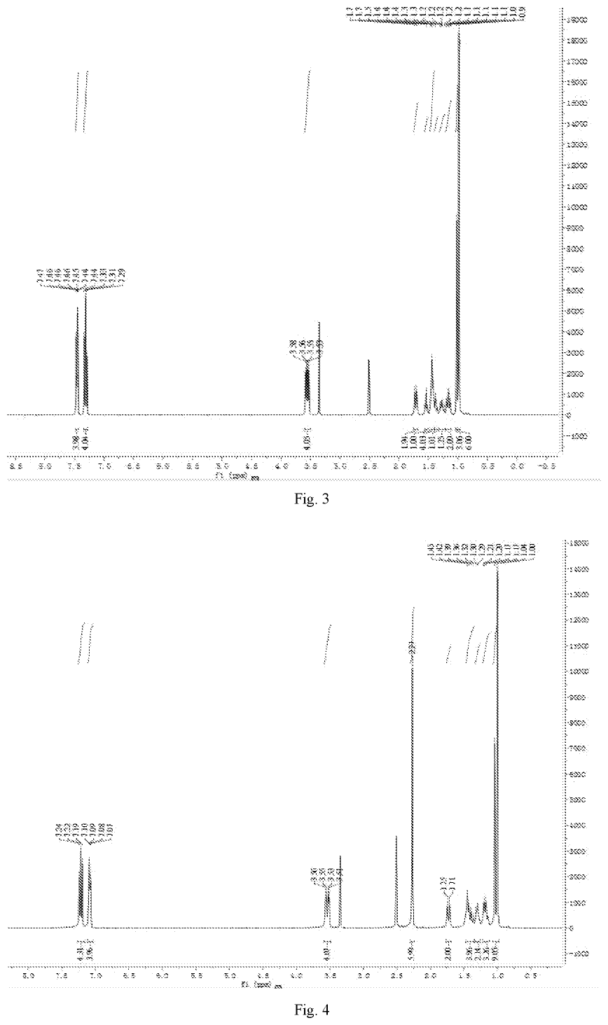

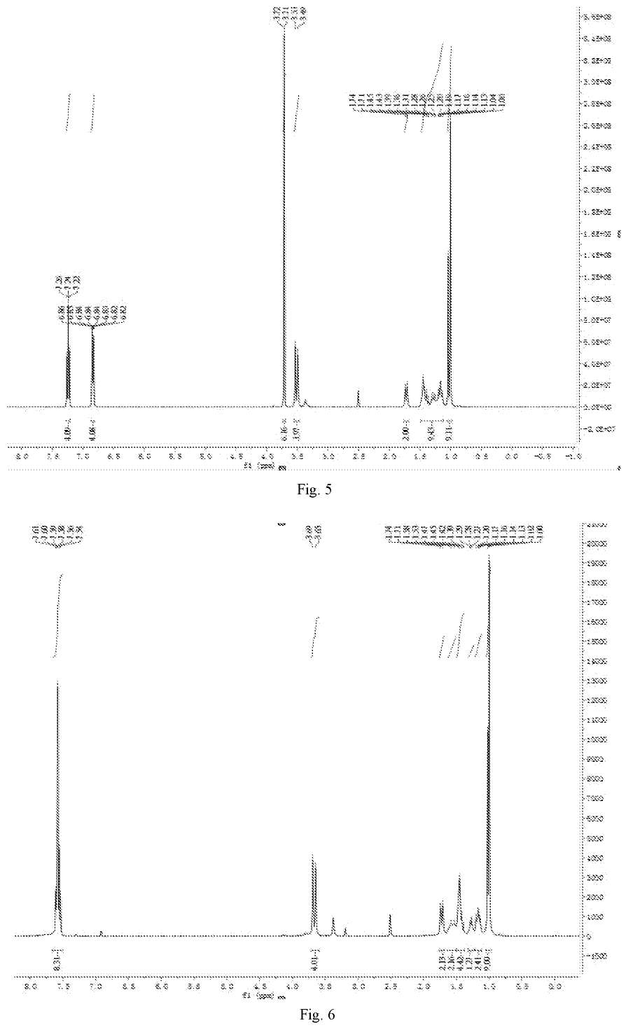

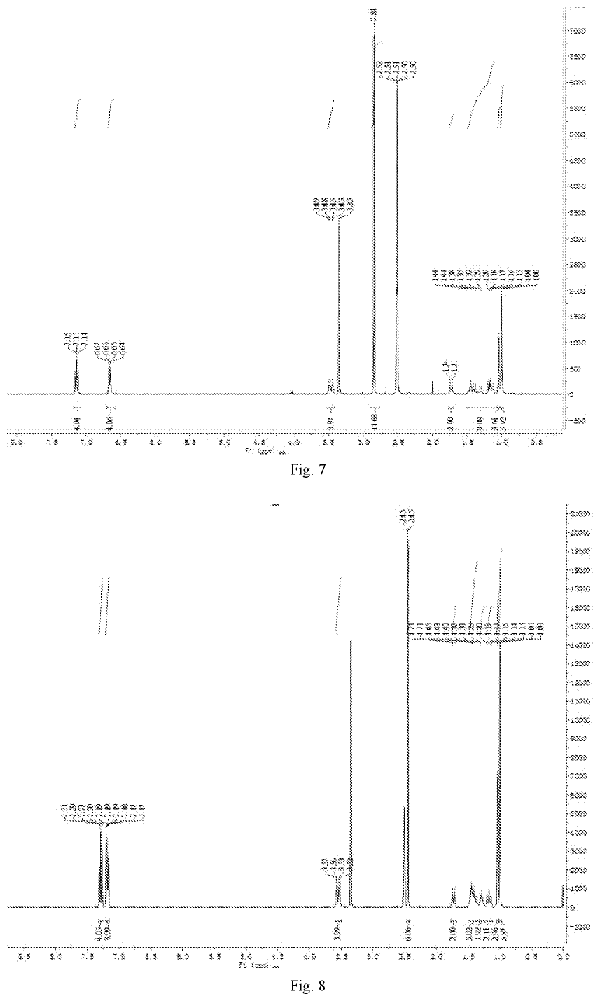

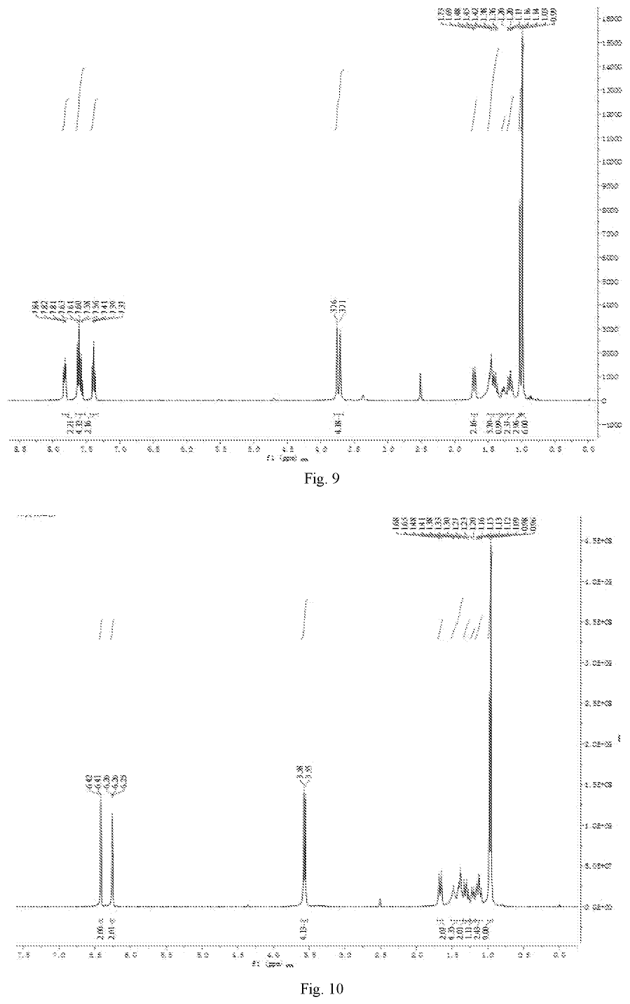

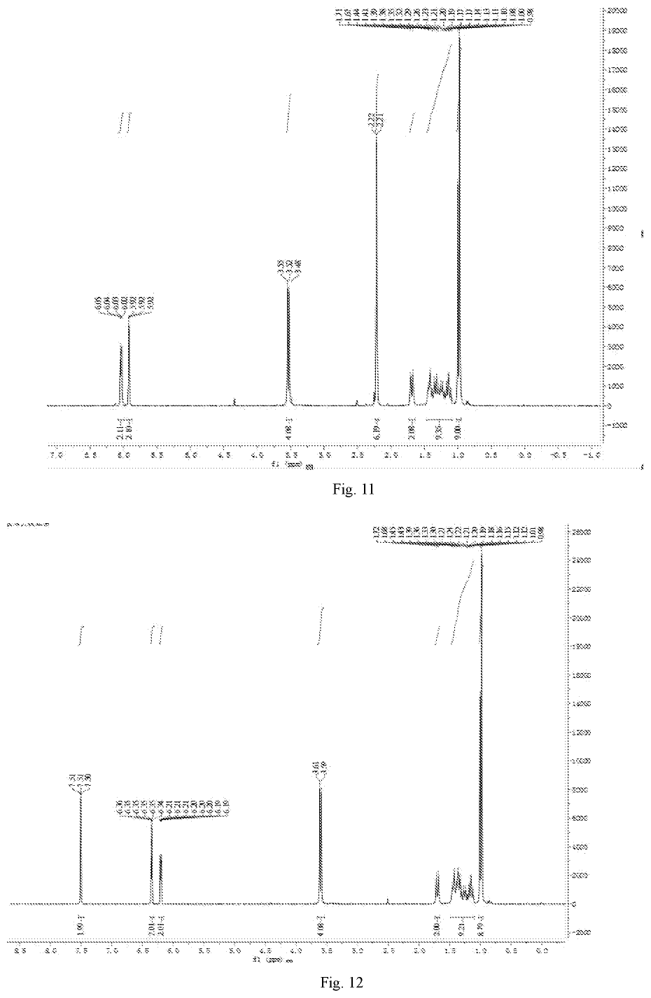

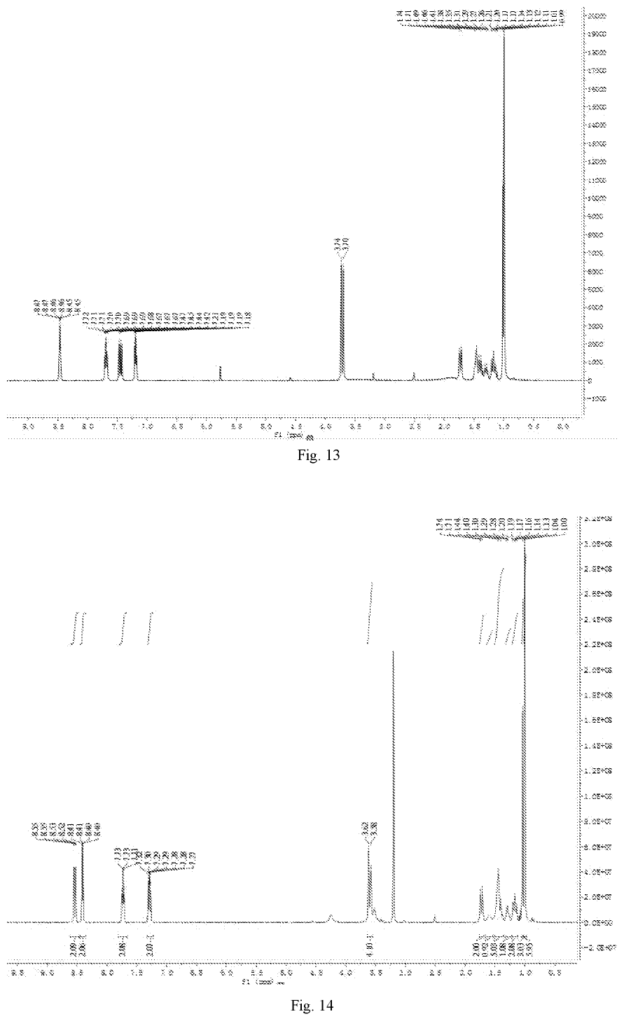

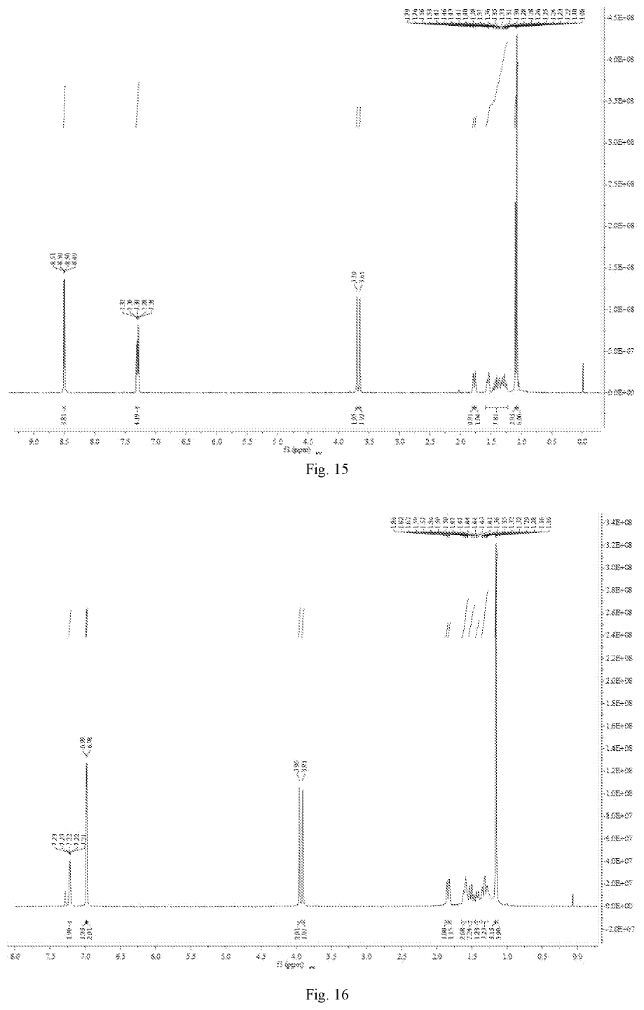

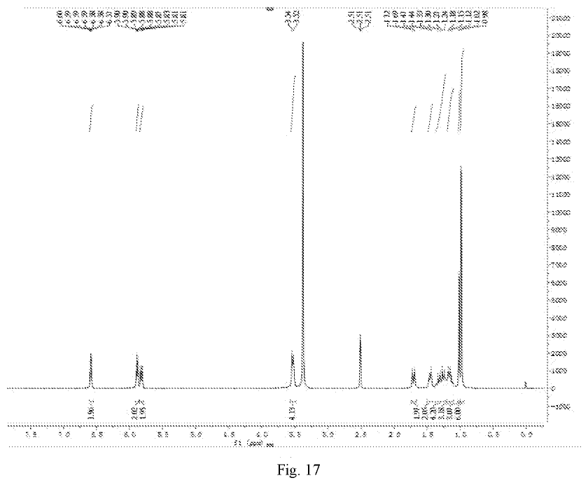

### DESCRIPTION OF EMBODIMENTS

Analytical Method

The product is analyzed with the gas phase peak area normalization method. The gas phase conditions: Shimadzu GC-2014AF, the carrier gas N₂ pressure is 0.6MPa, the air pressure is 0.6 MPa, H₂ pressure is 0.6 MPa; the programmed heating method is used, and the heating program is: 70° C. (kept for 2 min, rate 3° C./min)→130° C. (kept for 0 min, rate 10° C./min)→270° C. (kept for 2 min).

The present invention discloses cis-p-menthane-1,8-di-secondary amine compounds as well as their preparation methods and application in weed control. The preparation method of the compounds is to use cis-p-menthane-1,8-di-Schiff base compounds as the raw material and to reduce imine groups in the raw material into chemically stable secondary amine groups through the hydrogenation reduction reaction to get cis-p-menthane-1,8-di-secondary amine compounds. The present invention also uses the Petri dish seed germination method to determine the root and stem growth inhibition levels of barnyard grass incubated under different concentrations of cis-p-menthane-1,8-di-secondary amine compounds to evaluate the herbicidal activity of the compounds. The general structure of cis-p-menthane-1,8-di-secondary amine compounds is as follows:

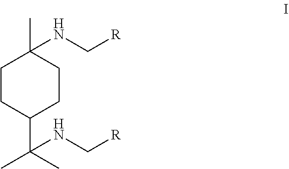

In general structure I, R is any one of

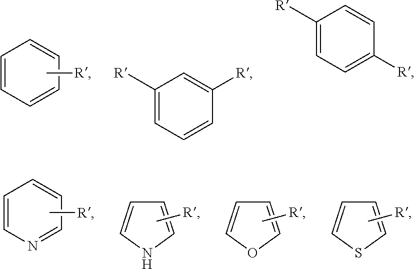

and R′ is any one of hydrogen, alkyl, substituted alkyl or halogen groups.

Step 1: Dissolve cis-p-menthane-1,8-di-Schiff base compounds in methanol, and add the reducing agent into the resulted solution in batches under the ice bath condition every half an hour. After a certain amount of reducing agent is added into the solution each time, the reaction will continue for some time. After the reaction is over, the reaction solution is quenched with distilled water and extracted with dichloromethane, and the resulted extract phase is then dried with anhydrous sodium sulfate, filtered and distilled under reduced pressure to obtain the crude cis-p-menthane-1,8-di-secondary amine compounds, which further undergo recrystallization or silica gel column chromatography to get pure cis-p-menthane-1,8-di-secondary amine compounds. The polar organic solvent is any one of methanol, ethanol, tetrahydrofuran, dimethylformamide and dimethyl sulfoxide; and the reducing agent is any one of sodium borohydride, potassium borohydride, lithium aluminum hydride, etc. The molar ratio of cis-p-menthane-1,8-di-Schiff base compounds to the reducing agent is between 1:1˜1:5; the reaction temperature is between −10˜50° C., and the reaction time is between 1˜24 h. The inhibitory effects of the synthesized compounds on normal cells are all between −10˜10%, proving that none of the related compounds has cytotoxicity.

Step 2: Accurately weigh 1 mmol cis-p-menthane-1,8-di-Schiff base compounds and cis-p-menthane-1,8-di-secondary amine compounds respectively, dissolve each of them with 0.25 mL DMF, and add a drop of Tween 80 in each resulted solution. After being fully dissolved, each solution is transferred to a 100 mL volumetric flask respectively and then diluted to the mark with distilled water to obtain a solution with a concentration of 10 mmol/L as the mother liquor. The two-fold dilution method is used to prepare diluted solutions with a series of concentrations (the concentrations of DMF and Tween 80 in the diluted solutions are the same as those in the mother liquor).

Barnyard grass seeds are placed in a 28° C. incubator and are soaked in an appropriate amount of distilled water for 12 hours there. After the clean water is filtered out, the seeds are then placed in the 28° C. incubator for 24 hours to germinate. Spread 2 pieces of filter paper in each of several Petri dishes, and place 10 barnyard grass seeds with the same size in each dish. Repeat each treatment for three times The dishes are divided into two groups: test dishes and control dishes. Add into each test dish 10 mL of the sample solution with the corresponding concentration of the target compound, and add into each control dish the same amount of the mixture of water, DMF and Tween 80 as the blank control. Add 10 seeds to each of the dishes (both test dishes and control dishes), place them in an artificial climate incubator, and incubate them for 96 hours under a temperature of 28° C., an illumination of 5000 1×, a photoperiod of day:night=16:8 and a relative humidity of 70-80%. The experimental data is processed and analyzed with DPS software to calculate the inhibition rate (y) of cis-p-menthane-1,8-di-secondary amine compounds on the root growth or stem growth of barnyard grass seeds.

$$y = \frac{{x}_{0} - {x}_{1}}{{x}_{0}}$$

Wherein, y is the inhibition rate of root growth or stem growth (%), x₀ is the root length or stem length of the control, and x₁ is the root length or stem length of the sample.

### EXPERIMENTAL EXAMPLE 1

Add 4.14 g (10 mmol) of cis-p-menthane-1,8-bis-(4-chlorophenyl) Schiff base (homemade. For the preparation method, please refer to the experimental example section of ZL201610942979.3) into a three-necked flask containing 20 mL methanol. Under the ice-water bath condition, add a total of 1.513 g (40 mmol) sodium borohydride into the flask in 3 times under magnetic stirring, with an interval of 30 min each time. After the addition is complete, the resulted mixture react at room temperature for 2 hours. After the reaction is complete, the resulted reaction solution is added with 20 mL of water to quench the reaction and then extracted with dichloromethane. The organic phase is collected, washed with saturated brine, dried with anhydrous sodium sulfate, and filtered with a filter. The solvent in the organic phase is then removed through vacuum distillation. The resulted solid is dissolved in ethanol and then re-crystallized in ethanol to obtain pure cis-p-menthane-1,8-bis-(4-chlorobenzyl) secondary amine. The yield is 85%.

Accurately weigh 1 mmol cis-p-menthane-1,8-bis-(4-chlorophenyl)Schiff base and cis-p-menthane-1,8-bis-(4-chlorobenzyl)amine respectively, dissolve each of them with 0.25 mL DMF, and add a drop of Tween 80 in each resulted solution. After being fully dissolved, each solution is transferred to a 100 mL volumetric flask respectively and then diluted to the mark with distilled water to obtain a solution with a concentration of 10 mmol/L as the mother liquor. The two-fold dilution method is used to prepare diluted solutions with a series of concentrations (5 mmol/L, 2.5 mmol/L, 1.25 mmol/L, 0.625 mmol/L, 0.3125 mmol/L and 0.1563 mmol/L; the concentrations of DMF and Tween 80 in the diluted solutions are the same as those in the mother liquor).

Barnyard grass seeds are placed in a 28° C. incubator and are soaked in an appropriate amount of distilled water for 12 hours there. After the clean water is filtered out, the seeds are then placed in a 30° C. incubator for 24 hours to germinate. Spread 2 pieces of filter paper in each Petri dish, and place 10 barnyard grass seeds with the same size in each dish. Repeat each treatment for three times The dishes are divided into two groups: test dishes and control dishes. Add into each test dish 10 mL of the sample solution with the above corresponding concentration of the target compound, and add into each control dish the same amount of the mixture of water, DMF and Tween 80 as the blank control. Add 10 seeds to each of the dishes (both test dishes and control dishes), place them in an artificial climate incubator, and incubate them for 96 hours under a temperature of 28° C., an illumination of 5000 1×, a photoperiod of day:night=16:8 and a relative humidity of 70-80%. The experimental data is processed and analyzed with DPS software. It is calculated that cis-p-menthane-1,8-bis-(4-chlorophenyl)Schiff base solutions with a concentration of 5 mmol/L, 2.5 mmol/L, 1.25 mmol/L, 0.625 mmol/L and 0.3125 mmol/L have an inhibition rate of 20.9%, 22.8%, 23.9%, 4.6% and 1.4% on the stem growth of barnyard grass seeds respectively, and an inhibition rate of 92.4%, 49.9%, 76.7%, 24.3% and −5.0% on the root growth of barnyard grass seeds respectively; and cis-p-menthane-1,8-bis-(4-chlorobenzyl)amine solutions with a concentration of 5 mmol/L, 2.5 mmol/L, 1.25 mmol/L, 0.625 mmol/L, 0.3125 mmol/L and 0.15625 mmol/L have an inhibition rate of 75.1%, 68.0%, 63.4%, 65.8%, 41.6% and 35.7% on the stem growth of barnyard grass seeds respectively, and an inhibition rate of 96.7%, 91.6%, 90.8%, 87.3%, 57.1% and 31.4% on the root growth of barnyard grass seeds respectively.

### EXPERIMENTAL EXAMPLE 2

Except that the raw material cis-p-menthane-1,8-di-Schiff base compound is cis-p-menthane-1,8-bis-(4-fluorobenzyl)Schiff base, other operational conditions and procedures in this experimental example are the same as those indicated in Experimental example 1. The target product is cis-p-menthane-1,8-bis-(4-fluorobenzyl)amine, and the yield rate is 98%. cis-p-Menthane-1,8-bis-(4-fluorobenzyl)Schiff base solutions with a concentration of 5 mmol/L, 2.5 mmol/L, 1.25 mmol/L, 0.625 mmol/L and 0.3125 mmol/L have an inhibition rate of 32.3%, 20.1%, 23.9%, 2.7% and −13.7% on the stem growth of barnyard grass seeds respectively, and an inhibition rate of 79.7%, 82.9%, 85.7%, 72.8% and 15.7% on the root growth of barnyard grass seeds respectively. cis-p-Menthane-1,8-bis-(4-fluorobenzyl)amine solutions with a concentration of 5 mmol/L, 2.5 mmol/L, 1.25 mmol/L, 0.625 mmol/L, 0.3125 mmol/L and 0.15625 mmol/L have an inhibition rate of 100.0%, 85.9%, 75.1%, 58.3%, 10.5% and 7.2% on the stem growth of barnyard grass seeds respectively, and an inhibition rate of 100.0%, 98.6%, 78.6%, 78.6%, 48.3% and 38.2% on the root growth of barnyard grass seeds respectively.

### EXPERIMENTAL EXAMPLE 3

Except that the raw material cis-p-menthane-1,8-di-Schiff base compound is cis-p-menthane-1,8-bis-(4-bromobenzyl)Schiff base, other operational conditions and procedures in this experimental example are the same as those indicated in Experimental example 1. The target product is cis-p-menthane-1,8-bis-(4-bromobenzyl)amine, and the yield rate is 96%. cis-p-Menthane-1,8-bis-(4-bromobenzyl)Schiff base solutions with a concentration of 5 mmol/L, 2.5 mmol/L, 1.25 mmol/L, 0.625 mmol/L and 0.3125 mmol/L have an inhibition rate of 55.9%, 39.3%, 42.3%, 19.6% and 6.5% on the stem growth of barnyard grass seeds respectively, and an inhibition rate of 69.4%, 72.6%, 46.8%, 28.5% and −3.2% on the root growth of barnyard grass seeds respectively. cis-p-Menthane-1,8-bis-(4-bromobenzyl)amine solutions with a concentration of 5 mmol/L, 2.5 mmol/L, 1.25 mmol/L, 0.625 mmol/L, 0.3125 mmol/L and 0.15625 mmol/L have an inhibition rate of 92.3%, 88.2%, 58.5%, 51.7%, 35.4% and 25.2% on the stem growth of barnyard grass seeds respectively, and an inhibition rate of 100.0%, 97.4%, 89.6%, 83.4%, 57.6% and 30.7% on the root growth of barnyard grass seeds respectively.

### EXPERIMENTAL EXAMPLE 4

Except that the raw material cis-p-menthane-1,8-di-Schiff base compound is cis-p-menthane-1,8-bis-(4-methylbenzyl)Schiff base, other operational conditions and procedures in this experimental example are the same as those indicated in Experimental example 1. The target product is cis-p-menthane-1,8-bis-(4-methylbenzyl)amine, and the yield rate is 97%. cis-p-Menthane-1,8-bis-(4-methylbenzyl)amine solutions with a concentration of 5 mmol/L, 2.5 mmol/L, 1.25 mmol/L, 0.625 mmol/L, 0.3125 mmol/L and 0.15625 mmol/L have an inhibition rate of 93.3%, 87.1%, 74.7%, 65.9%, 64.6% and 42.1% on the stem growth of barnyard grass seeds respectively, and an inhibition rate of 100.0%, 95.2%, 91.9%, 89.6%, 86.6% and 42.5% on the root growth of barnyard grass seeds respectively.

### EXPERIMENTAL EXAMPLE 5

Except that the raw material cis-p-menthane-1,8-di-Schiff base compound is cis-p-menthane-1,8-bis-(4-methoxybenzyl)Schiff base, other operational conditions and procedures in this experimental example are the same as those indicated in Experimental example 1. The target product is cis-p-menthane-1,8-bis-(4-methoxybenzyl)amine, and the yield rate is 94%. cis-p-Menthane-1,8-bis-(4-methoxybenzyl) Schiff base solutions with a concentration of 5 mmol/L, 2.5 mmol/L, 1.25 mmol/L, 0.625 mmol/L and 0.3125 mmol/L have an inhibition rate of 2.1%, 8.0%, 4%, 6.9% and 15.4% on the stem growth of barnyard grass seeds respectively, and an inhibition rate of 7.6%, 28.1%, 55.3%, 66.7% and 70.7% on the root growth of barnyard grass seeds respectively. cis-p-Menthane-1,8-bis-(4-methoxybenzyl)amine solutions with a concentration of 5 mmol/L, 2.5 mmol/L, 1.25 mmol/L, 0.625 mmol/L, 0.3125 mmol/L and 0.15625 mmol/L have an inhibition rate of 84.3%, 84.4%, 70.6%, 72.5%, 27.9% and 9.9% on the stem growth of barnyard grass seeds respectively, and an inhibition rate of 100.0%, 96.5%, 95.1%, 88.7%, 42.2% and 37.8% on the root growth of barnyard grass seeds respectively.

### EXPERIMENTAL EXAMPLE 6

Except that the raw material cis-p-menthane-1,8-di-Schiff base compound is cis-p-menthane-1,8-bis-(4-trifluoromethylbenzyl)Schiff base, other operational conditions and procedures in this experimental example are the same as those indicated in Experimental example 1. The target product is cis-p-menthane-1,8-bis-(4-trifluoromethylbenzyl)amine, and the yield rate is 96%. cis-p-Menthane-1,8-bis-(4-trifluoromethylbenzyl)Schiff base solutions with a concentration of 5 mmol/L, 2.5 mmol/L, 1.25 mmol/L, 0.625 mmol/L and 0.3125 mmol/L have an inhibition rate of 31.1%, 19.5%, 14.6%, 30.9% and 20.6% on the stem growth of barnyard grass seeds respectively, and an inhibition rate of 48.4%, 7.1%, 17.5%, 0.8% on the root growth of barnyard grass seeds respectively. cis-p-Menthane-1,8-bis-(4-trifluoromethylbenzyl)amine solutions with a concentration of 5 mmol/L, 2.5 mmol/L, 1.25 mmol/L, 0.625 mmol/L, 0.3125 mmol/L and 0.15625 mmol/L have an inhibition rate of 78.5%, 83.4%, 67.0%, 55.1%, 42.2% and 11.6% on the stem growth of barnyard grass seeds respectively, and an inhibition rate of 100.0%, 100.0%, 86.1%, 82.3%, 62.8% and 1.0% on the root growth of barnyard grass seeds respectively.

### EXPERIMENTAL EXAMPLE 7

Except that the raw material cis-p-menthane-1,8-di-Schiff base compound is cis-p-menthane-1,8-bis-(4-dimethylaminobenzyl)Schiff base, other operational conditions and procedures in this experimental example are the same as those indicated in Experimental example 1. The target product is cis-p-menthane-1,8-bis-(4-dimethylaminobenzyl)amine, and the yield rate is 92%. cis-p-Menthane-1,8-bis-(4-dimethylaminobenzyl)Schiff base solutions with a concentration of 5 mmol/L, 2.5 mmol/L, 1.25 mmol/L, 0.625 mmol/L, 0.3125 mmol/L have an inhibition rate of 13.2%, 1.9%, 6.9%, 9.5% and −12.2% on the stem growth of barnyard grass seeds respectively, and an inhibition rate of 67.1%, 59.3%, 49.8%, 45.4% and 9.7% on the root growth of barnyard grass seeds respectively. cis-p-Menthane-1,8-bis-(4-dimethylaminobenzyl)amine solutions with a concentration of 5 mmol/L, 2.5 mmol/L, 1.25 mmol/L, 0.625 mmol/L, 0.3125 mmol/L and 0.15625 mmol/L have an inhibition rate of 56.6%, 34.3%, 22.3%, 24.9%, 19.6% and 19.1% on the stem growth of barnyard grass seeds respectively, and an inhibition rate of 89.3%, 70.3%, 48.1%, 21.0%, 31.0% and 14.4% on the root growth of barnyard grass seeds respectively.

### EXPERIMENTAL EXAMPLE 8

Except that the raw material cis-p-menthane-1,8-di-Schiff base compound is cis-p-menthane-1,8-bis-(4-methylthiobenzyl)Schiff base, other operational conditions and procedures in this experimental example are the same as those indicated in Experimental example 1. The target product is cis-p-menthane-1,8-bis-(4-methylthiobenzyl)amine, and the yield rate is 89%. cis-p-Menthane-1,8-bis-(4-methylthiobenzyl)amine solutions with a concentration of 5 mmol/L, 2.5 mmol/L, 1.25 mmol/L, 0.625 mmol/L, 0.3125 mmol/L and 0.15625 mmol/L have an inhibition rate of 85.1%, 77.5%, 83.4%, 66.2%, 66.0% and 22.8% on the stem growth of barnyard grass seeds respectively, and an inhibition rate of 98.5%, 96.0%, 90.8%, 77.2%, 58.1% and 32.0% on the root growth of barnyard grass seeds respectively.

### EXPERIMENTAL EXAMPLE 9

Except that the raw material cis-p-menthane-1,8-di-Schiff base compound is cis-p-menthane-1,8-bis-(2-trifluoromethylbenzyl)Schiff base, other operational conditions and procedures in this experimental example are the same as those indicated in Experimental example 1. The target product is cis-p-menthane-1,8-bis-(2-trifluoromethylbenzyl)amine, and the yield rate is 83%. cis-p-Menthane-1,8-bis-(2-trifluoromethylbenzyl)Schiff base solutions with a concentration of 5 mmol/L, 2.5 mmol/L, 1.25 mmol/L, 0.625 mmol/L and 0.3125 mmol/L have an inhibition rate of 21.2%, 12.1%, 17.7%, 13.2% and 9% on the stem growth of barnyard grass seeds respectively, and an inhibition rate of 34.8%, 20.6%, 2.7%, 15.4% and 20.9% on the root growth of barnyard grass seeds respectively. cis-p-Menthane-1,8-bis-(2-trifluoromethylbenzyl)amine solutions with a concentration of 5 mmol/L, 2.5 mmol/L, 1.25 mmol/L, 0.625 mmol/L, 0.3125 mmol/L and 0.15625 mmol/L have an inhibition rate of 90.3%, 87.4%, 59.6%, 49.0%, 43.6% and 36.9% on the stem growth of barnyard grass seeds respectively, and an inhibition rate of 100%, 100%, 93. %, 68.0%, 57.0% and 51.9% on the root growth of barnyard grass seeds respectively.

### EXPERIMENTAL EXAMPLE 10

Except that the raw material cis-p-menthane-1,8-di-Schiff base compound is cis-p-menthane-1,8-bis-(5-bromofuran-2)-Schiff base, other operational conditions and procedures in this experimental example are the same as those indicated in Experimental example 1. The target product is cis-p-menthane-1,8-bis-(5-bromofuran-2-methyl)amine, and the yield rate is 88%. cis-p-Menthane-1,8-bis-(5-bromofuran-2-methyl)amine solutions with a concentration of 5 mmol/L, 2.5 mmol/L, 1.25 mmol/L, 0.625 mmol/L, 0.3125 mmol/L and 0.15625 mmol/L have an inhibition rate of 71.8%, 50.5%, 30.3%, 11.6%, 5.6% and 10.1% on the stem growth of barnyard grass seeds respectively, and an inhibition rate of 90.7%, 84.4%, 61.5%, 54.8%, 39.6% and 42.2% on the root growth of barnyard grass seeds respectively.

### EXPERIMENTAL EXAMPLE 11

Except that the raw material cis-p-menthane-1,8-di-Schiff base compound is cis-p-menthane-1,8-bis-(5-methylfuran)Schiff base, other operational conditions and procedures in this experimental example are the same as those indicated in Experimental example 1. The target product is cis-p-menthane-1,8-bis-(5-methylfuran-2-methyl)amine, and the yield rate is 87%. cis-p-Menthane-1,8-bis-(5-methylfuran-2-methyl)amine solutions with a concentration of 5 mmol/L, 2.5 mmol/L, 1.25 mmol/L, 0.625 mmol/L, 0.3125 mmol/L and 0.15625 mmol/L have an inhibition rate of 79.8%, 36.5%, 17.1%, 6.8%, 12.5% and 7.2% on the stem growth of barnyard grass seeds respectively, and an inhibition rate of 95.5%, 64.4%, 50.3%, 24.7%, 41.3% and 23.1% on the root growth of barnyard grass seeds respectively.

### EXPERIMENTAL EXAMPLE 12

Except that the raw material cis-p-menthane-1,8-di-Schiff base compound is cis-p-menthane-1,8-bis(furan-2)-Schiff base, other operational conditions and procedures in this experimental example are the same as those indicated in Experimental example 1. The target product is cis-p-menthane-1,8-bis(furan-2-methyl)amine, and the yield rate is 88%. cis-p-Menthane-1,8-bis(furan-2-methyl)amine solutions with a concentration of 5 mmol/L, 2.5 mmol/L, 1.25 mmol/L, 0.625 mmol/L, 0.3125 mmol/L and 0.15625 mmol/L have an inhibition rate of 65.3%, 41.1%, 19.3%, 16.1%, 8.6% and 0% on the stem growth of barnyard grass seeds respectively, and an inhibition rate of 92.7%, 72.7%, 48.8%, 39.0%, 36.3% and 0% on the root growth of barnyard grass seeds respectively.

### EXPERIMENTAL EXAMPLE 13

Except that the raw material cis-p-menthane-1,8-di-Schiff base compound is cis-p-menthane-1,8-bis(pyridine-2)-Schiff base, other operational conditions and procedures in this experimental example are the same as those indicated in Experimental example 1. The target product is cis-p-menthane-1,8-bis(pyridine-2-methyl)amine, and the yield rate is 90%. cis-p-Menthane-1,8-bis(pyridine-2-methyl)amine solutions with a concentration of 5 mmol/L, 2.5 mmol/L, 1.25 mmol/L, 0.625 mmol/L, 0.3125 mmol/L and 0.15625 mmol/L have an inhibition rate of 88.0%, 65.3%, 46.6%, 30.1%, 26.4% and 23.8% on the stem growth of barnyard grass seeds respectively, and an inhibition rate of 100.0%, 95.8%, 61.0%, 33.8%, 20.5% and 18.9% on the root growth of barnyard grass seeds respectively.

### EXPERIMENTAL EXAMPLE 14

Except that the raw material cis-p-menthane-1,8-di-Schiff base compound is cis-p-menthane-1,8-bis(pyridine-3)-Schiff base, other operational conditions and procedures in this experimental example are the same as those indicated in Experimental example 1. The target product is cis-p-menthane-1,8-bis(pyridine-3-methyl)amine, and the yield rate is 81%. cis-p-Menthane-1,8-bis(pyridine-3-methyl)amine solutions with a concentration of 5 mmol/L, 2.5 mmol/L, 1.25 mmol/L, 0.625 mmol/L, 0.3125 mmol/L and 0.15625 mmol/L have an inhibition rate of 84.9%, 65.3%, 44.6%, 41.5%, 30.6% and 38.0% on the stem growth of barnyard grass seeds respectively, and an inhibition rate of 95.2%, 86.1%, 41.4%, 19.2%, 10.2% and 5.7% on the root growth of barnyard grass seeds respectively.

### EXPERIMENTAL EXAMPLE 15

Except that the raw material cis-p-menthane-1,8-di-Schiff base compound is cis-p-menthane-1,8-bis(pyridine-4)-Schiff base, other operational conditions and procedures in this experimental example are the same as those indicated in Experimental example 1. The target product is cis-p-menthane-1,8-bis(pyridine-4-methyl)amine, and the yield rate is 91%. cis-p-Menthane-1,8-bis(pyridine-4-methyl)amine solutions with a concentration of 5 mmol/L, 2.5 mmol/L, 1.25 mmol/L, 0.625 mmol/L, 0.3125 mmol/L and 0.15625 mmol/L have an inhibition rate of 86.7%, 71.3%, 53.5%, 32.7%, 34.5% and 28.6% on the stem growth of barnyard grass seeds respectively, and an inhibition rate of 96.5%, 91.2%, 71.5%, 40.6%, 37.9% and 19.0% on the root growth of barnyard grass seeds respectively.

### EXPERIMENTAL EXAMPLE 16

Except that the raw material cis-p-menthane-1,8-di-Schiff base compound is cis-p-menthane-1,8-bis(thiophen-2)-Schiff base, other operational conditions and procedures in this experimental example are the same as those indicated in Experimental example 1. The target product is cis-p-menthane-1,8-bis(thiophen-2-methyl)amine, and the yield rate is 86%. cis-p-Menthane-1,8-bis(thiophen-2-methyl)amine solutions with a concentration of 5 mmol/L, 2.5 mmol/L, 1.25 mmol/L, 0.625 mmol/L, 0.3125 mmol/L and 0.15625 mmol/L have an inhibition rate of 59.1%, 51.0%, 45.3%, 39.2%, 28.9% and 13.3% on the stem growth of barnyard grass seeds respectively, and an inhibition rate of 86.0%, 75.1%, 50.0%, 5.8%, 1.5% and 0.8% on the root growth of barnyard grass seeds respectively.

### EXPERIMENTAL EXAMPLE 17

Except that the raw material cis-p-menthane-1,8-di-Schiff base compound is cis-p-menthane-1,8-bis(pyrrole-2)-Schiff base, other operational conditions and procedures in this experimental example are the same as those indicated in Experimental example 1. The target product is cis-p-menthane-1,8-bis(pyrrole-2-methyl)amine, and the yield rate is 65%. cis-p-Menthane-1,8-bis(pyrrole-2-methyl)amine solutions with a concentration of 5 mmol/L, 2.5 mmol/L, 1.25 mmol/L, 0.625 mmol/L, 0.3125 mmol/L and 0.15625 mmol/L have an inhibition rate of 78.3%, 71.5%, 48.2%, 28.6%, 26.5% and 15.4% on the stem growth of barnyard grass seeds respectively, and an inhibition rate of 94.4%, 91.9%, 93.1%, 87.7%, 60.5% and 1.7% on the root growth of barnyard grass seeds respectively.

### EXPERIMENTAL EXAMPLE 18

Except that the molar ratio of cis-p-menthane-1,8-bis-(4-chlorobenzyl)Schiff base to sodium borohydride is 1:1, other operational conditions and procedures in this experimental example are the same as those indicated in Experimental example 1. The yield rate is 77%.

### EXPERIMENTAL EXAMPLE 19

Except that the molar ratio of cis-p-menthane-1,8-bis-(4-chlorobenzyl)Schiff base to sodium borohydride is 1:5, other operational conditions and procedures in this experimental example are the same as those indicated in Experimental example 1. The yield rate is 85%.

### EXPERIMENTAL EXAMPLE 20

Except that the reaction temperature is 25° C., other operational conditions and procedures in this experimental example are the same as those indicated in Experimental example 1. The yield rate is 82%.

### EXPERIMENTAL EXAMPLE 21

Except that the reaction time is 0.5 h, other operational conditions and procedures in this experimental example are the same as those indicated in Experimental example 1. The yield rate is 80%.

### EXPERIMENTAL EXAMPLE 22

Except that the reaction time is 3 h, other operational conditions and procedures in this experimental example are the same as those indicated in Experimental example 1. The yield rate is 85%.

### EXPERIMENTAL EXAMPLE 23

Except that the reducing agent is potassium borohydride, other operational conditions and procedures in this experimental example are the same as those indicated in Experimental example 1.

### EXPERIMENTAL EXAMPLE 24

Except that the reducing agent is lithium aluminum hydride, other operational conditions and procedures in this experimental example are the same as those indicated in Experimental example 1.

1. A type of cis-p-menthane-1,8-di-secondary amine compounds, which is characterized by: the general structure is as follows: 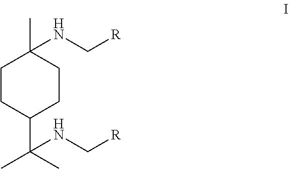
In general structure I, R is any one of 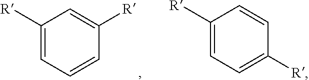
R′ is any one of alkyl, substituted alkyl or halogen groups; or
R is selected from 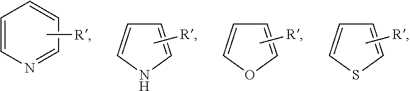
and R′ is any one of hydrogen, alkyl, substituted alkyl or halogen groups; or
R is 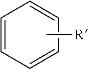
and R′is selected from hydrogen and halogen groups excluding Cl.

2. The preparation method of cis-p-menthane-1,8-di-secondary amine compounds mentioned in claim 1 is characterized by: use cis-p-menthane-1,8-di-Schiff base compounds as the raw material, dissolve the raw material in a polar organic solvent and then add a reducing agent into the resulted solution in batches to have reduction reaction; after the reaction finishes, the reaction solution is subject to post-treatment to obtain a pure product.

3. The preparation method of cis-p-menthane-1,8-di-secondary amine compounds mentioned in claim 2 is characterized by: the above-mentioned polar organic solvent is any one of methanol, ethanol, tetrahydrofuran, dimethylformamide and dimethyl sulfoxide, or a mixture of several of them.

4. The preparation method of cis-p-menthane-1,8-di-secondary amine compounds mentioned in claim 2 is characterized by: the above-mentioned reducing agent is any one of sodium borohydride, potassium borohydride and lithium aluminum hydride.

5. The preparation method of cis-p-menthane-1,8-di-secondary amine compounds mentioned in claim 2 is characterized by: the molar ratio of cis-p-menthane-1,8-di-Schiff base compounds to the reducing agent is between 1:1-5.

6. The preparation method of cis-p-menthane-1,8-di-secondary amine compounds mentioned in claim 2 is characterized by: the reaction temperature is between −10˜50° C.

7. The preparation method of cis-p-menthane-1,8-di-secondary amine compounds mentioned in claim 2 is characterized by: the reaction time is 1˜24 h.

8. The preparation method of cis-p-menthane-1,8-di-secondary amine compounds mentioned in claim 2 is characterized by: the above-mentioned post-treatment specifically refers to quenching the reaction solution with distilled water, extracting it with dichloromethane, drying the extract phase with anhydrous sodium sulfate, filtering the extract phase, obtaining crude cis-p-menthane-1,8-di-secondary amine compounds through distillation under reduced pressure, and obtaining the pure product through recrystallization or silica gel column chromatography.

9. The cis-p-menthane-1,8-di-secondary amine compounds mentioned in claim 1 are used as active ingredients of herbicides.

10. The cis-p-menthane-1,8-di-secondary amine compounds mentioned in claim 1 are used as active ingredients of herbicides for controlling barnyard grass in paddy fields.
